(19) United States
(12) Patent Application Publication
(10) Pub. No.: US 2005/0194005 A1
(43) Pub. Date: Sep. 8, 2005

(54) Children's nasal spray containers and inhalers

(76) Inventors: Patricia J. Berube, Cohasset, MA (US); Amy L. White, Hanover, MA (US)

Correspondence Address: Karl F. Milde, Jr., Esq.;MILDE, HOFFBERG & MACKLIN, L.L.P., Suite 460, 10 Bank Street, White Plains, NY, 10606, US

(21) Appl. No.: 10/892,585

(22) Filed: Jul. 16, 2004

Related U.S. Application Data
(60) Provisional application No. 60/550,397, filed on Mar. 5, 2004.

(51) Int. Cl. A61M 11/00

(52) U.S. Cl. 128/200.14

(57) ABSTRACT

A container for dispensing nasal spray, ear drops, or an inhalant mist, formed in the three-dimensional shape of an entertaining figurine, such as an elephant, or a toy, having appendages which serve as handles for grasping and manipulating the container to administer the spray or mist to a child.

### FIELD OF THE INVENTION

The present invention relates to containers for dispensing nasal sprays, ear drops and nasal mist inhalant, and specifically to containers which are “user-friendly” for children.

### BACKGROUND OF THE INVENTION

Common dispensing containers for nasal sprays include the squeeze bottle containers, and the pump action containers. Examples of the squeeze bottle nasal spray containers may be seen in U.S. Pat. No. 4,513,891 and also the references cited therein. Typically, these containers comprise a nozzle fitted to the top of a container having flexible side walls, and containing a reservoir of fluid to be dispensed, and means, such as a dip tube for conveying the fluid to the nozzle when the side walls of the container are squeezed.

During dispensing of the spray, the nozzle is disposed directly under, or within, the nostril while the container is squeezed to dispense the fluid spray into the nostril. The precise positioning, and coordinated movement required to dispense the spray is considered too complicated to be performed by a child. While it may be easy for an adult to use these dispensers on themselves, it is much more difficult for an adult to administer the spray to a child.

A pump action container typically includes a hard walled container, and a horizontally extending finger platform at the bottom of the nozzle. The throat of the container is disposed up into the nozzle, such as by placing the opposed thumb on the bottom of the container and pushing towards the fingers, to dispense a fluid spray from the nozzle. A spring action returns the container to its original position, with the throat beneath the finger platform, ready for its next use.

Through the years each type of container has been improved to produce uniform, reproducible, fine sprays of fluid. However, the precise positioning and coordinated movement required to use these devices has not changed. In addition, little attempt has been need to design a container which is “user-friendly” for children.

Similarly, inhalers have been developed to carefully, and reproducibly produce a fine mist of fluid droplets to be inhaled through the mouth into the bronchial tubes. Typically, the inhaler includes a pump action container which dispenses the spray into a breathing tube, at right angles to the pumping action. The other end of the breathing tube is placed within the mouth prior to actuating the pump. Using the fingers and opposed thumb to pump the container, a fine mist is released into the breathing tube, to be instantly inhaled by the patient. As with the nasal spray containers, careful placement and coordinated movement is required to use these devices. And again, little attempt has been made to design an inhaler which is “user-friendly” for children.

### SUMMARY OF THE INVENTION

The present invention comprises a nasal spray dispensing container, nasal inhaler, or ear drop dispenser fashioned to assist an adult in administering the nasal spray, bronchial mist or ear drops to a child by configuring the container as an entertaining three-dimensional figurine, or toy. The containers of the present invention distract a child from the task of administering a fluid, such as a pharmaceutical preparation, by providing an alternative focus; items of tactile and visual interest; and a reminder of a toy, or a play stimulus.

At the same time, the container is provided with multiple handles. These handles enable the adult to administer the spray, mist or drops to a child, and assist an older child learn to use such a dispensing container. When an adult administers the spray, mist or drops to a child, the nostril, mouth or ear may be a moving target. Thus the task of positioning the spray of mist container, and the coordinated movement to dispense the fluid are vastly complicated. In addition, though an adult using such a dispenser on themselves positions and squeezes/pumps in front of their own face, when administering to a child, both the positioning and the coordinated movement to dispense may have to be performed at awkward angles and positions. The many handles on the containers of the present invention aid the adult in maintaining a grasp on the container, and moving it within their fingers, to be able to perform the coordinated movement to dispense the spray or mist.

Likewise, when administering ear drops to a child, any means, such as watching or holding the ear drop container, may keep their ears from moving, and thereby assist the adult in administering the drops. In addition, when the ear drop container is in the shape of an animal figurine, making the head of the figurine into a side-hinged, “flip-top”, which exposes the dropper, also places the head of the figurine in a mimicking, “ear-up” position, required to dispense ear drops. Then, if the child mimic the head position of the figurine, it will be easier to administer the drops. Similarly, the front-hinged head of the figurine on a nasal spray container could place the nostrils of the animal down, the position needed to properly administer nasal spray.

The three-dimensional figurine may be formed about, configured into, or configured to contain replaceable units of, a squeeze bottle nasal spray dispensing container, a pump action nasal spray dispensing container, a pump action inhaler, an ear drop container with dispenser, or any future construction or spray or mist dispenser. The appendages of the figurine, such as the legs, ears, tail, wings, etc. of an animal figure, may provide additional handles, or means for grasping, holding, and manipulating the container. In addition, the figurine of the container may be provided with fine fingering details, to interest the small fingers of a child. Graphic details of the figurine, such as its eyes, spots, and its overall coloring may serve as means of visual interest for the child. The three dimensional figurine may be an animal, such as an elephant, giraffe, or bear, or a fantasy figure, such as a unicorn or a dragon.

If desired, a sounding device may be incorporated into the dispensing containers, such that dispensing the spray or mist is preceded by a sound, such as an elephant trumpeting. When using such a container, the child may be encouraged to inhale when they hear the sound.

It is an object of the present invention to provide a container for dispensing a nasal spray, ear drops or an inhalant mist, which is easy to use with a child. It is a further object of the present invention to provide a nasal spray container, or an inhaler, which decreases the fear of the child, and engages the interest of the child. It is get a further object of the invention to provide a nasal spray container, or and inhaler, which elicits the participation of the child in receiving the spray or mist.

These objects, as well as other objects which will become apparent from the discussion that follows, are achieved, in accordance with the present invention which comprises a container for dispensing nasal spray, ear drops or bronchial mist, which container comprises an entertaining, three dimensional figurine of an animal or a toy object, having multiple handles formed of, e.g., the appendages of the animals, and foci of tactile and visual interest, and perhaps aural interest, to distract a child during administration of the mist, and encourage their participation in receiving the spray or mist.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a side view of a nasal spray dispensing container according to the present invention, in the shape of an elephant.

FIG. 2 is a side view of unicorn shaped nasal spray dispensing container according to the present invention.

FIG. 2A is a side view of a unicorn dispensing ear drop container, wherein the ear drop containing vessel is encased in a unicorn figurine, with the head and unicorn forming the dropper top.

FIG. 3 is a side view of a giraffe shaped nasal spray dispensing container according to the present invention.

FIG. 3A is a side view of giraffe container for dispensing nasal spray, where the head of the giraffe forms a “flip-top”, hinged at the front, such that when the top is flipped, the dispensing opening is exposed for use, and the nostrils of the giraffe are facing down.

FIG. 4 is a side view of a dragon shaped nasal spray dispensing container according to the present invention.

FIG. 5 is a side view of an inhalant mist dispensing container according to the present invention, shaped like a panda bear.

FIG. 5A is a side view of another embodiment of an inhalant mist dispensing container, wherein the head of the figurine is a “flip-top”, which, when opened, exposes the plunger top of an inhalant dispensing canister.

FIG. 6 is a front view of another embodiment of an ear drop dispensing container wherein the head of the figurine is a “flip-top” hinged at the side, such that the head is turned on its side, placing the ear of the figurine up, and exposing the dropper top for dispensing the ear drops contained therein.

### DESCRIPTION OF THE PREFERRED EMBODIMENTS

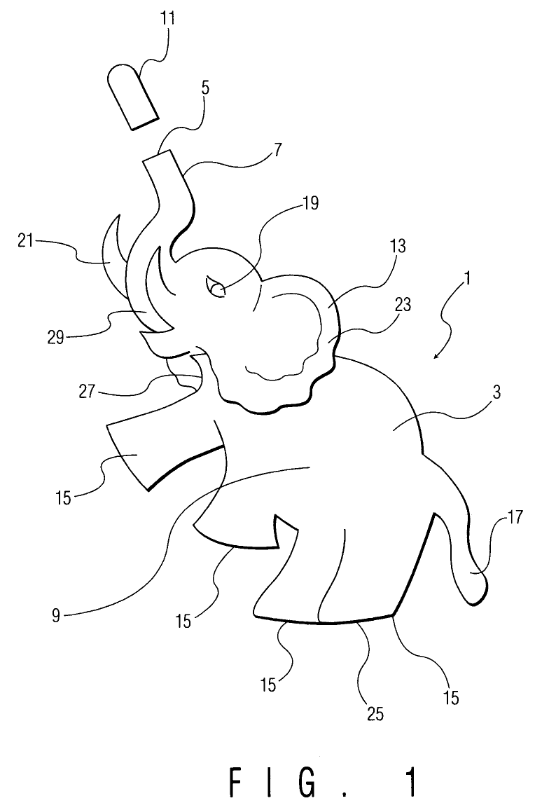

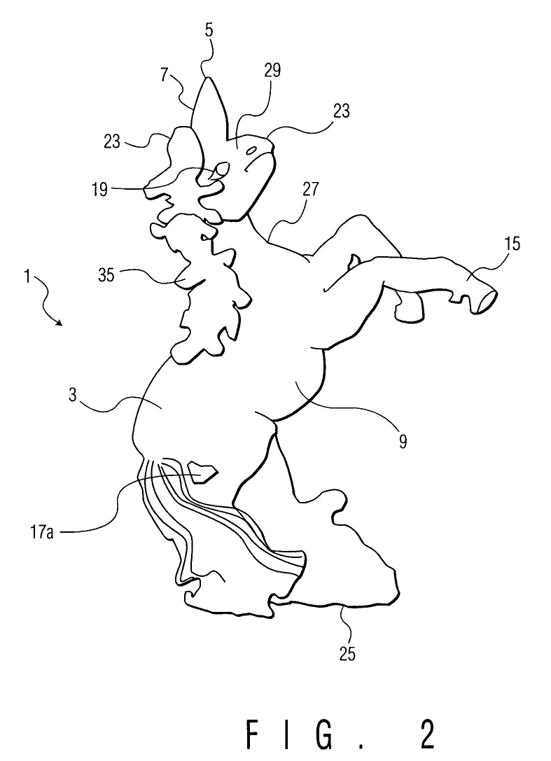

The preferred embodiments of the present invention will now be described with reference to FIGS. 1-6 of the drawings. Identical elements in the various figures are designated with the same reference numerals.

FIG. 1 illustrates a container for dispensing nasal spray, shown generally at 1. The container is formed in the shape of three-dimensional figure, 3, in this embodiment, an elephant. In this particular embodiment, the container is formed such that the sprayed dispensing opening, or nozzle, 5, is formed from the trunk of the elephant. The end of the trunk comprises the insertion region, 7, which may be disposed within the nostril of the child before dispensing the spray. When constructed as a squeeze bottle dispensing container, the container may be squeezed in the activation region, 9, roughly positioned at the center of the flexible side walls of the container. If desired, a sanitary cap, 11, may be provided for the dispensing opening, or nozzle, 5, and the insertion region, 7. The appendages of the elephant, in this embodiment the ears, 13, feet, 15, and tail, 17, provide additional grasping handles for the person administering the nasal spray from the container. They may also provide in regions of fingering interest for the child to whom the nasal spray is being administered. Other finer details of the elephant figurine, such as a tusk, 21, provide further regions of fingering interest for the small exploring fingers of a child. Graphic detail, such as the eyes, 19, provides visual interest for the child.

Alternatively, the elephant figurine container need be configured as a pump action nasal spray dispenser. When so configured, the ears may be extended away from the body of the elephant, to form a finger platform, 23. The hind feet may comprise the thumb placement surface, 25. Displacement of the thumb towards the finger platform may telescope, e.g., the throat, 27, into the head, 29, to dispense the nasal spray from the opening, 5.

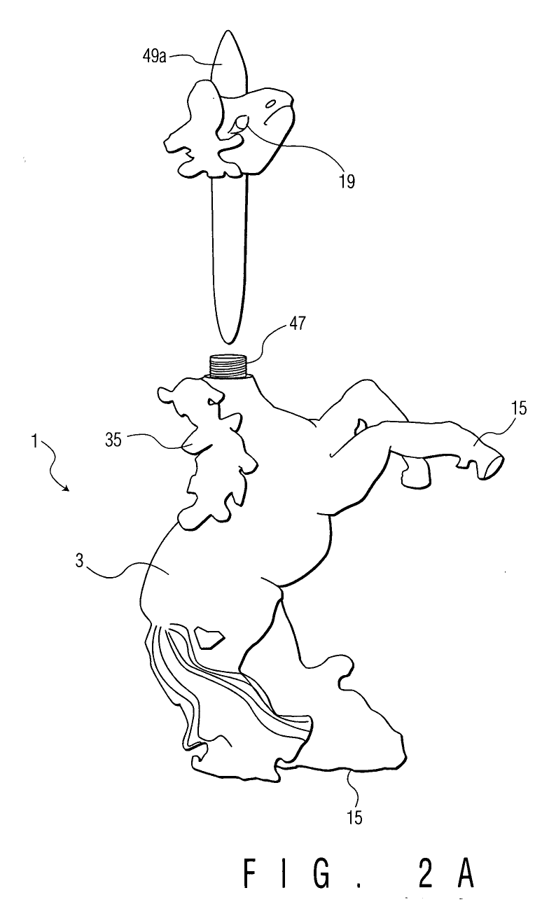

FIG. 2 illustrates an alternative embodiment of nasal spray dispensing container of the present invention, 1, formed in the three-dimensional shape, 3, of a unicorn. In this embodiment that dispensing opening, 5, is formed at the tip of the horn, with the insertion region, 7, comprising the end of the horn, as shown. In the embodiment shown, the mane, 35, forms a fine fingering detail for the child, as does the tail, 17a. When configured as a pump action dispenser, the snout, 29, and the top of the mane, may form the finger platform, 23, and hind feet form the thumb placement region, 25.

FIG. 2A depicts a dropper-dispensing container, 1, for a medicament, such as ear drops. A vessel, such as a glass bottle is encased in the entertaining figurine, 3, which has appendages for grasping the container, fine finger detail, and graphic detail, as described above. In this particular embodiment, the head of the figurine contains a dropper cap, 49a, for dispensing e.g., ear drops. As shown in FIG. 2A, when the dropper cap is unscrewed from the neck of the bottle,47, the dropper cap may be used to dispense the ear drops.

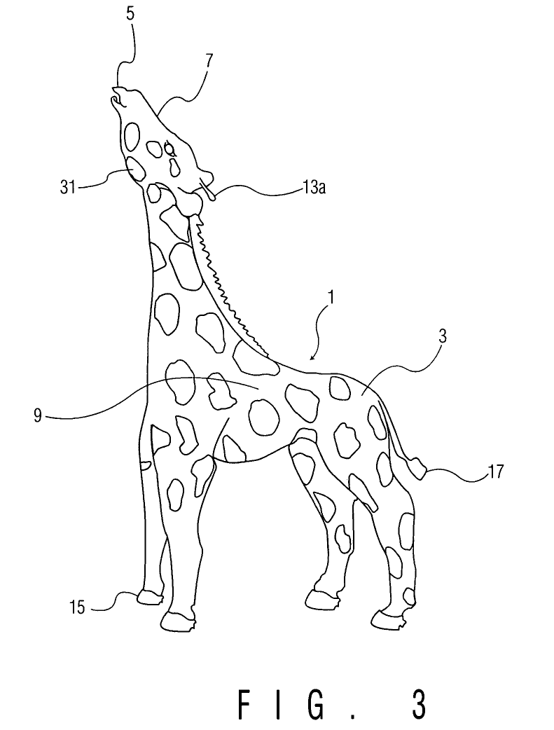

In FIG. 3, the nasal spray dispensing container, 1, is formed in the three-dimensional shape, 3, of a giraffe, with the snout and mouth forming of the insertion region, 7, and the dispensing opening, 5, respectively. The spots, 31, form graphic detail of visual interest the child, and horny protuberances, 13a, near the ears, form fine fingering detail.

FIG. 3A depicts an alternative embodiment of a nasal spray dispensing container according to the present invention. In this embodiment, the dispensing opening, 5, is not the head, or top, of the figurine, but the head of the figurine is removable to expose the dispensing opening, 5. In this particular embodiment, the head is formed as a front-hinged, “flip-top cap, 39, which both moves, but remains attached to the container, by means of hinge, 41. In other embodiments, the head may be completely removable, and re-fastenable, such as a snap-on cap. However, the front-hinged, “flip-top” head not only remains attached, but the front hinge disposes the nostrils of the animal figurine in a nose down position, generally perpendicular to the ground, (note arrow), exactly the position recommended for the administration of nasal spray.

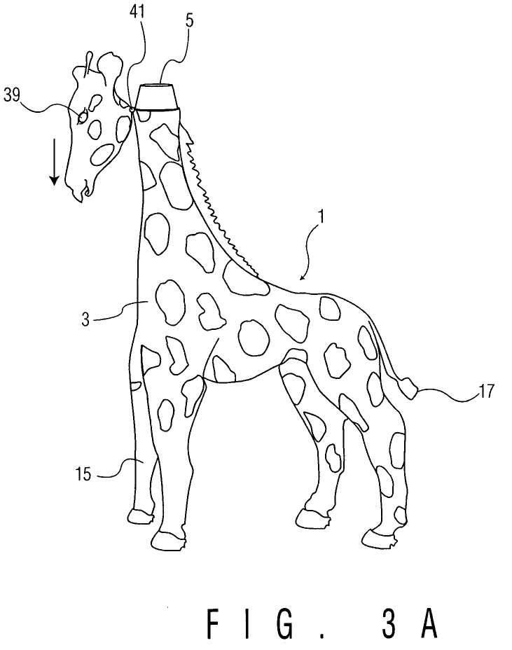

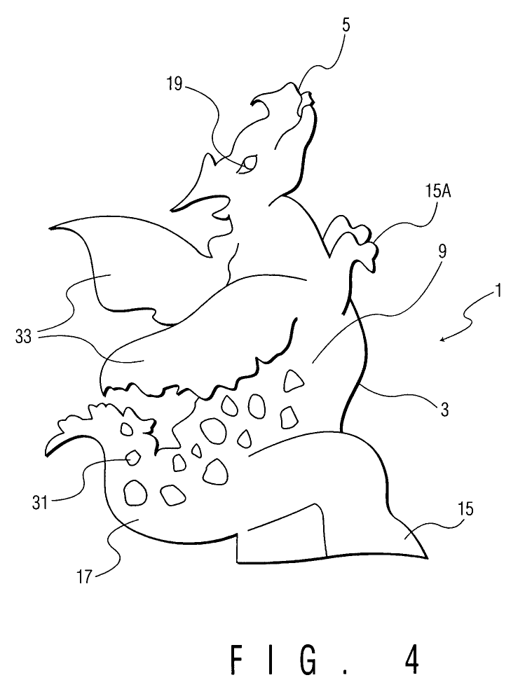

FIG. 4 illustrates a nasal spray dispensing container, 1, formed in the three-dimensional shape, 3, of a dragon. As shown in this embodiment, the front feet, 15a, present fine fingering detail for the child, but only small handles for the user. Graphic detail includes the overall coloring, as well as, e.g., the spots, 31, and eyes, 19. The wings, 33, present not only additional grasping handles for the adult administering the spray, but additional fingering detail for the child.

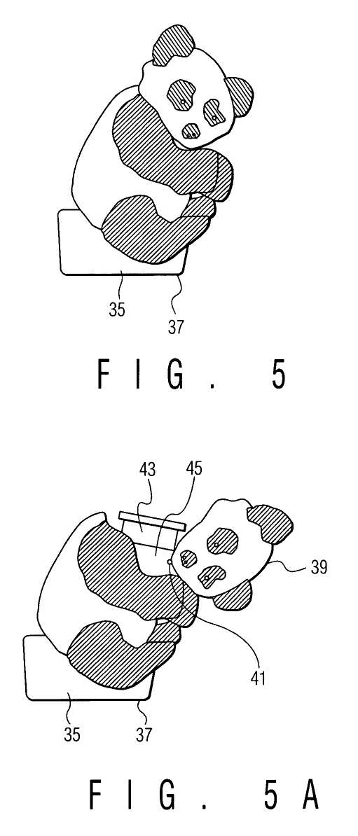

As shown in FIG. 5, a bronchial inhaler may also be formed in the shape of an entertaining figurine, or toy. As shown, the pump action container is disclosed within a three-dimensional figure, 3, of a Panda bear. The bear sits atop the breathing, or inhaling, tube, 35. During use, the free end of the breathing/inhaling tube is placed in the mouth, and the inhalant is dispensed by placing the thumb on the remote end of the inhaling tube, 37, and one or more fingers on the bear's head, and pushing down on the head to activate the release of the inhalant mist into the tube, 35.

FIG. 5A is an alternative embodiment of the inhalant mist container of FIG. 5, with a “flip-top” head exposing the plunger, 43, of the inhalant mist canister, 45. As in the embodiment illustrated in FIG. 3A, the hinge, 41, permits movement, but retains the head cap, 39 of the figurine attached to the body of the figurine. In this embodiment, the canister may be removable and replaceable within the figurine container. In this particular embodiment, the person administering the mist pushes the plunger, not the head of the figurine. This embodiment avoids wear and tear of the head of the figurine, and presents a generally flat-topped plunger, which may be easier to press than the head of the figurine.

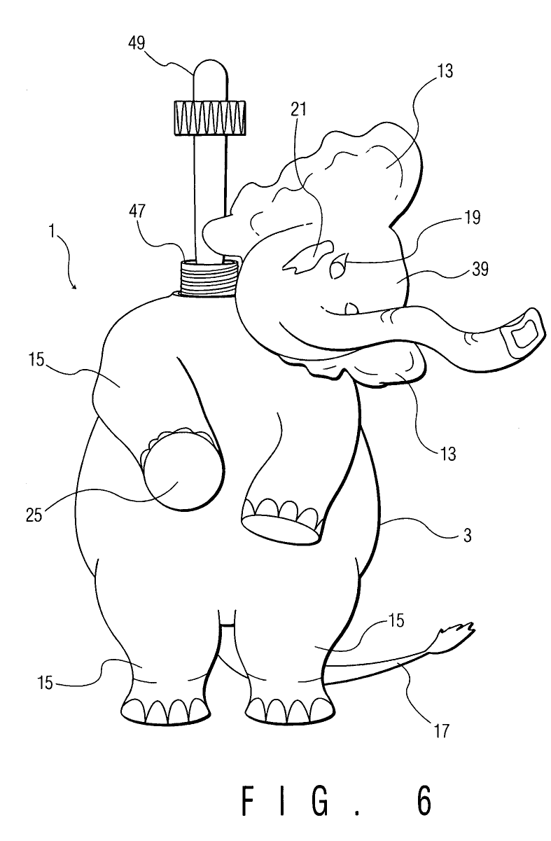

FIG. 6 illustrated another hinged head cap container according to the present invention. In this particular embodiment, the container is adapted to dispense ear drops from dropper, 49. As shown, the head of the figurine is side-hinged, so that when flipped about the hinge, the head rotates to the side, placing the ear of the figurine in an ear-up position, mimicking the position the child must assume to have the drops administered into the ear. Flipping the head about the hinge (not shown), also exposes the dropper, 49, which may be removed from the medicament vessel, in this embodiment, unscrewed from the treaded neck of the bottle, 47. When the child tilts his/her head, and looks the elephant in the eye, they will be ready to receive the ear drops.

Forming the nasal spray dispensing containers, or inhalers, in the shape of an animal, or other entertaining or toy figure, serves a number of purposes. First, the containers of the present invention distract the child from the task at hand, providing a focus of alternative interest. Additionally, forming the nasal spray dispensing container, or inhalers, are intended to provide visual and tactile pleasure, and perhaps instigate play, and cooperation. In addition, the containers provide multiple handles to assist the adult administrator in maintaining a grasp on, and manipulating the position or the container, to accomplish the positioning and coordinated movement required to dispense the spray or mist. In addition, the “flip-top” head may be configured and hinged to mimic the head position of the child during administration of the medicament.

There has thus been shown and described a novel container for a nasal spray, or an inhaler, which fulfills all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A nasal spray dispensing container comprising an entertaining figurine, containing a vessel for nasal spray, a dispensing opening, and means for dispensing the nasal spray through the dispensing opening.

2. A squeeze bottle nasal spray dispensing container wherein the container is formed in the shape of an entertaining figurine.

3. The nasal spray dispensing container of claim 2, further comprising additional means for grasping the container, comprising the appendages of the figurine.

4. The nasal spray dispensing container of claim 2, further comprising fingering details of the figurine.

5. The nasal spray dispensing container of claim 2, further comprising graphic details of the figurine.

6. The nasal spray dispensing container of claim 1, wherein the figurine comprises a three dimensional unicorn with appendages selected from the group consisting the head, legs, feet, tail and mane, which provide handles for grasping the container.

7. The nasal spray dispensing container of claim 1, wherein the figurine comprises a three dimensional elephant having appendages selected from the group consisting the head, ears, legs, feet, tail, truck and tusks, which provide handles for grasping the container, and wherein the dispensing opening is disposed in the trunk.

8. The nasal spray dispensing container of claim 1, wherein the figurine comprises a three dimensional giraffe having appendages selected from the group consisting the head, neck, horny protuberances near the ears, legs, feet, and tail, which provide handles for grasping the container, and wherein the dispensing opening is disposed in the head.

9. The nasal spray dispensing container of claim 1, wherein the figurine comprises a three dimensional dragon having appendages selected from the group consisting the head, wings, legs, feet, and tail, which provide handles for grasping the container, wherein the dispensing opening is disposed in the head.

10. A pump action nasal spray dispensing container comprising the shape of an entertaining figurine, containing a vessel for nasal spray, a dispensing opening, and means for dispensing the nasal spray through the dispensing opening.

11. The nasal spray dispensing container of claim 10, further comprising additional means for grasping the container, comprising the appendages of the figurine.

12. The nasal spray dispensing container of claim 10, further comprising fingering details of the figurine.

13. The nasal spray dispensing container of claim 10, further comprising graphic details of the figurine.

14. A container for an inhaler, said container comprising an entertaining figurine.

15. A pump action inhalant mist dispenser comprising the three dimensional shape of an entertaining figurine, containing a canister for the inhalant, an inhaling tube, and means for dispensing a mist of the inhalant.

16. The pump action inhalant dispenser of claim 15, further comprising additional means for grasping the container, comprising the appendages of the figurine.

17. The pump action inhalant dispenser of claim 15, further comprising fingering details of the figurine.

18. The pump action inhalant dispenser of claim 15, further comprising graphic details of the figurine.

19. A container for an inhaler, said container comprising a panda bear figurine.

20. A container for an inhaler, said container comprising an animal figurine.

21. A nasal spray container as in claim 2, wherein the figurine has a head, separable from the body of the figurine, said head covering the dispensing opening.

22. A nasal spray dispensing container as in claim 21, further comprising a hinge, said head being attached the body by the hinge.

23. A nasal spray container as in claim 22, wherein the figurine depicts an animal with nostrils, and the hinge is attached to the front of the head of the figurine, such that when the head is moved about the hinge, exposing the dispensing opening, the nostrils of the figurine are perpendicular to the ground.

24. A container for ear drops, said container comprising an entertaining figurine containing a vessel for the ear drops, a removeable but replaceable vessel cap, and a dropper.

25. A container for ear drops as in claim 24, wherein the figurine comprises an animal having appendages selected from the group consisting of the head, legs, feet, neck and tail, which provide handles for grasping and holding the container.

26. A container for ear drops as in claim 24, wherein the figurine has a head, separable from the body of the figurine, said head covering the cap and dropper.

27. A container for ear drops as in claim 24, further comprising a hinge, said head being attached the body by the hinge.

28. A container for ear drops as in claim 27, wherein hinge is placed at the side of the head, such that when the head is rotated about the hinge, exposing the cap and stopper, the head of the figurine is generally perpendicular to the body.

29. The container for ear drops as in claim 27, wherein the figurine has ears, and the ears face up when the head of the figurine is rotated about the hinge.
